SEQUENCE LISTING (25 sequences)

SEQ ID NO: 1 - protein, 449 aa, synthetic construct
MPPLLAPLLCLALLPALAARGPRCISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEDLGPGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 1350 bp, synthetic construct
  source 1..1350: mol_type = other DNA
atgccgccgctcctggcgcccctgctctgcctggcgctgctgcccgcgctcgccgcacgaggcccgcgatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatctgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggatctgggcccgggcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 3 - protein, 455 aa, synthetic construct
MQPPSLLLLLLLLLLCVSVVRPLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQDLGPGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1368 bp, synthetic construct
  source 1..1368: mol_type = other DNA
atgcagcccccttcactgctgctgctgctgctgctgctgctgctatgtgtctcagtggtcagacccttggaagacatgtgtctgagccagccgtgccatggggatgcccaatgcagcaccaaccccctcacaggctccacactctgcctgtgtcagcctggctattcggggcccacctgccaccaggacctggacgagtgtctgatggcccagcaaggcccaagtccctgtgaacatggcggttcctgcctcaacactcctggctccttcaactgcctctgtccacctggctacacaggctcccgttgtgaggctgatcacaatgagtgcctctcccagccctgccacccaggaagcacctgtctggacctacttgccaccttccactgcctctgcccgccaggcttagaagggcagctctgtgaggtggagaccaacgagtgtgcctcagctccctgcctgaaccacgcggattgccatgacctgctcaacggcttccagtgcatctgcctgcctggattctccggcacccgatgtgaggaggatatcgatgagtgcagaagctctccctgtgccaatggtgggcagtgccaggaccagcctggagccttccactgcaagtgtctcccaggctttgaagggccacgctgtcaagatctgggcccgggcgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 5 - protein, 212 aa, Homo sapiens
MPPLLAPLLCLALLPALAARGPRCISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCE

SEQ ID NO: 6 - protein, 218 aa, Homo sapiens
MQPPSLLLLLLLLLLCVSVVRPLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQ

SEQ ID NO: 7 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 24 aa, synthetic construct
EPKSSDKTHTCPPCPAPELLGGPS

SEQ ID NO: 9 - protein, 24 aa, synthetic construct
EPKSSDKTHTCPPCPAPELLGGSS

SEQ ID NO: 10 - protein, 24 aa, synthetic construct
EPKSSGSTHTCPPCPAPELLGGSS

SEQ ID NO: 11 - protein, 19 aa, synthetic construct
DKTHTCPPCPAPELLGGPS

SEQ ID NO: 12 - protein, 19 aa, synthetic construct
DKTHTCPPCPAPELLGGSS

SEQ ID NO: 13 - protein, 208 aa, Homo sapiens
VFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 5 aa, synthetic construct
DLGPG

SEQ ID NO: 15 - protein, 12 aa, synthetic construct
GSGSGSGSGSGS

SEQ ID NO: 16 - protein, 7 aa, synthetic construct
AGGGGSG

SEQ ID NO: 17 - protein, 8 aa, synthetic construct
AGGGGSGG

SEQ ID NO: 18 - protein, 8 aa, synthetic construct
QPDEPGGS

SEQ ID NO: 19 - protein, 17 aa, synthetic construct
ELQLEESAAEAQDGELD

SEQ ID NO: 20 - protein, 6 aa, synthetic construct
TVAAPS

SEQ ID NO: 21 - protein, 9 aa, synthetic construct
QPDEPGGSG

SEQ ID NO: 22 - protein, 18 aa, synthetic construct
ELQLEESAAEAQDGELDG

SEQ ID NO: 23 - protein, 7 aa, synthetic construct
TVAAPSG

SEQ ID NO: 24 - protein, 194 aa, Homo sapiens
ARGPRCISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCE

SEQ ID NO: 25 - protein, 196 aa, Homo sapiens
LEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQ

(19) United States
(12) Patent Application Publication
(10) Pub. No.: US 2025/0223332 A1
(43) Pub. Date: Jul. 10, 2025

(54) NOTCH1 AND NOTCH4 DECOYS AND METHODS OF USE

(71) Applicant: THE BOARD OF TRUSTEES OF THE UNIVERSITY OF ILLINOIS, Urbana, IL (US)

(72) Inventors: Timothy SARGIS, Chicago, IL (US); Jan KITAJEWSKI, Chicago, IL (US)

(21) Appl. No.: 18/850,718

(22) PCT Filed: Mar. 24, 2023
(86) PCT No.: PCT/US2023/064896

§ 371 Date: Sep. 25, 2024

Related U.S. Application Data
(60) Provisional application No. 63/326,316, filed on Apr. 1, 2022.

(51) Int. Cl. C07K 14/705; A61K 38/17; A61P 9/00; A61P 27/02; C12N 15/62

(52) U.S. Cl. CPC C07K 14/705; A61K 38/177; A61P 9/00; A61P 27/02; C12N 15/62

(57) ABSTRACT

A fusion protein composed of Notch1 or Notch4 EGF-like repeats 10-14 fused to human IgG Fc is described. Also provided is a pharmaceutical composition including the fusion protein and use of the fusion protein in methods of inhibiting angiogenesis and treating a disease or disorder.

### INTRODUCTION

This application claims the benefit of priority from U.S. Provisional Application Ser. No. 63/326,316, filed Apr. 1, 2022, the contents of which are incorporated herein by reference in their entireties.

This invention was made with government support under grant number W81XWH-18-1-0019 awarded by the Department of Defense and grant number HL112626 awarded by the National Institutes of Health. The government has certain rights in this invention.

### REFERENCE TO AN ELECTRONIC SEQUENCE LISTING

The contents of the electronic sequence listing (name: UIC0103WO_ST26.xml; size: 27, 516 bytes; and date of creation: Mar. 9, 2023) is herein incorporated by reference in its entirety.

### BACKGROUND

Angiogenesis is a tightly regulated multi-step process that defines the development of new blood vessels emanating from existing vessels. Under physiological conditions, this action is confined to embryonic and post-natal development as well as tissue growth and wound healing during the adult life. Angiogenesis can be driven by hypoxia, inflammation, and other physiological and pathophysiological signals. Angiogenic aberrations have been implicated in several pathologies such as in cancer, vascular malformations, and retinopathy. Understanding how angiogenesis contributes to tumor growth and inflammation has long been a key area of interest for therapeutic intervention. In the absence of vascular support, tumors rarely develop past 2 mm³, demonstrating the critical role angiogenesis plays in the development of tumor growth. This vulnerability in turn pressures tumors to up-regulate pro-angiogenic factors and recruit nearby endothelial cells to maintain growth, and ultimately, metastatic spread.

Notch signaling functions in angiogenesis through the regulation of endothelial cell-fate decisions, often controlling separate angiogenic pathways, such as vascular endothelial growth factor receptor (VEGFR) signaling, to carry out this function. Mammals express four homologous notch receptors (Notch1-4) and five ligands (Jagged (JAG) 1, 2 and delta-like ligand (DLL) 1, 3, 4). Ligand-receptor interactions cause subsequent cleavage of the Notch receptor and translocation of the Notch intracellular domain (NICD) to the nucleus, leading to transcription of downstream target genes. It has been well-documented that the Notch ligand DLL4 acts through endothelial Notch as a negative regulator of VEGF receptors to restrict angiogenesis, thus producing an appropriate number of functional vessels. The Notch ligand, JAG1, plays a pro-angiogenic role but the mechanisms of JAG1-Notch signaling in endothelial cells are not well understood. Tumor vasculature regularly exploits angiogenic pathways that respond to hypoxia-regulated VEGF, which in turn up-regulates DLL4 that then Notch activates signaling. Thus, the involvement of Notch signaling in pathological angiogenesis intersects with VEGFR signaling and has marked it as a potential target for controlling this process. Further, Notch ligands can promote metastatic spread of tumor cells through both blood and lymphatic vascular systems.

Approaches to globally inhibit the activation of the Notch pathway have raised safety concerns due to toxicity. The most prominent class of Notch inhibitors are those that target γ-secretase. γ-Secretase inhibitors (GSI) block the cleavage of Notch and the subsequent translocation of the intracellular domain of Notch (ICN) to the nucleus, inhibiting Notch signaling. Aberrant activation of Notch1 in T cell acute lymphoblastic leukemia (T-ALL) patients led to the deployment of GSIs for use in clinical trials. However, most patients suffered from gastrointestinal symptoms such as diarrhea in a dose-dependent manner, making treatment sub-optimal. Animal studies have further confirmed that systemic inhibition of Notch signaling results in gastrointestinal toxicity due to accumulation of secretory goblet cells in the intestine. The development of DLL4 neutralizing antibodies was a promising next step in targeting the endothelial Notch1 signaling axis without the toxicity issues associated with global Notch blockade. The role of DLL4/Notch1 signaling in the development of the tumor vasculature have been studied extensively where it has been shown that Tip Cell formation, the initial cellular step of angiogenesis, is inhibited by DLL4-Notch signaling. Thus, the rationale to inhibit DLL4-Notch1 signaling appears paradoxical as its inhibition decreases tumor growth by triggering excessive but poorly perfused tumor vessels. Despite its promise, anti-DLL4 therapy evaluated in pre-clinical animal models has resulted in pathological changes in the liver as well as severe vascular neoplasms.

Development of new and safe approaches for targeting the Notch pathway remains a critical clinical problem, based on the potential to inhibit several types of malignancies by restricting functional vessels. Notch decoys including Notch1 (N1₁₋₃₆), repeats 1-24 (N1₁₋₂₄), or repeats 1-13 (N1₁₋₁₃) fused in frame with human IgG constant fragment (Fc) and epidermal growth factor (EGF)-like repeats 10-36 of human Notch1 (N1₁₀₋₃₆), repeats 14-36 (N1₁₄₋₃₆), repeats 10-24 (N1₁₀₋₂₄), or repeats 14-24 (N1₁₄₋₂₄) fused in frame with human IgG Fc have been described and shown to effectively inhibit tumor growth with minimal gastrointestinal toxicities associated with Notch inhibition (Kangsamaksin et al. (2015) Cancer Discovery 5 (2): 182-197). Adenoviral administration of decoy-producing vectors was used to produce and evaluate the activity of these Notch decoys in mice. While adenoviral vectors have been approved for some human trials, purified proteins provide control over dosage and carries less risk of inappropriate immune responses than with viral administration.

EGF-repeats of the Notch4 receptor protein operably affixed to a half-life-increasing moiety are described in U.S. Pat. No. 7,662,919. Notch1 decoy fusion proteins composed of EGF-like repeat 10-18 or 10-20 conjugated to the Fc portion of the human antibody is described in U.S. Pat. No. 11,026,996. However, these fusions are difficult to manufacture in sufficient amounts for use as therapeutic agents due to the number of elaborate post-translational modifications. Accordingly, there is an ongoing need for the development of therapeutically-useful Notch decoys that can be readily manufactured on a useful scale.

### SUMMARY OF THE INVENTION

This invention provides a fusion protein, the sequence of which, commencing at the N-terminus of the fusion protein, is identical to the sequence of amino acids in:
(a) an extracellular domain of a human Notch1 or Notch4 receptor protein, followed by
(b) an Fc portion of an antibody,
wherein the extracellular domain of the human Notch1 or Notch4 receptor protein
   (i) commences with the amino acid present at the N-terminus of EGF-like repeat 10 and
   (ii) extends to and includes the C-terminal amino acid of EGF-like repeat 14 as the C-terminal amino acid of the extracellular domain. In some aspects, the fusion protein further includes a signal sequence. A nucleic acid molecule encoding the fusion protein and a host cell harboring the nucleic acid molecule are also provided as is a pharmaceutical composition including the fusion protein in admixture with a pharmaceutically acceptable excipient.

The invention also provides the use of the fusion protein in methods for inhibiting angiogenesis and treating an angiogenic disease or disorder in a subject. In some aspects, the angiogenic disease or disorder comprises an ocular disease or disorder, e.g., age-related macular degeneration (AMD) or diabetic retinopathy. In other aspects, the angiogenic disease or disorder is cancer or vascular inflammation.

### BRIEF DESCRIPTION OF THE DRAWINGS

FIG. 1 shows the construction and expression of Notch decoys. Notch decoys are composed of specific EGF-like repeats (10-14) of the Notch1 or Notch4 extracellular domain fused to the human Fc domain. The resulting fusion proteins are referred to herein as “N1₁₀₋₁₄Fc” and “N4₁₀₋₁₄Fc.”

FIG. 2 shows that N1₁₀₋₁₄Fc inhibits DLL4-induced cleavage of the Notch1 receptor. HUVECs were plated onto 1 μg/mL of recombinant hDLL4 in the presence of N1₁₀₋₁₄Fc or IgG Fc isotype control for 24 hours and quantitated for cleaved Notch1 by western blot analysis.

FIG. 3 shows quantitative real-time polymerase chain reaction (qRT-PCR) analysis of targets of canonical Notch signaling in HUVECs treated with N1₁₀₋₁₄Fc. Cells were treated with either Notch peptibody or IgG Fc at indicated concentrations for 24 hours. ***P value <0.001, **P value <0.01, *P value <0.05.

FIG. 4 shows qRT-PCR analysis of targets of canonical Notch signaling in HUVECs treated with N4₁₀₋₁₄Fc. Cells were treated with either Notch peptibody or IgG Fc at indicated concentrations for 24 hours. *P value <0.05.

FIG. 5 shows mean sprout number of HUVEC-coated beads embedded in fibrin gel and treated with increasing doses of human IgG Fc, N1₁₀₋₁₄Fc, or N4₁₀₋₁₄Fc for 12 days. After 12 days, N1₁₀₋₁₄Fc significantly reduced sprout number at dosages of 5 and 10 μg/ml. Box-and-whisker plots show median, minimum, and maximum values.

FIG. 6 shows mean sprout length of HUVEC-coated beads embedded in fibrin gel and treated with increasing doses of human IgG Fc, N1₁₀₋₁₄Fc, or N4₁₀₋₁₄Fc for 12 days. After 12 days, N1₁₀₋₁₄Fc significantly reduced sprout length at dosages of 5 and 10 μg/ml. Box-and-whisker plots show median, minimum, and maximum values. **P value <0.01, *P value <0.05.

FIG. 7 shows that N1₁₀₋₁₄FC inhibits retinal angiogenesis in murine neonates. C57BL/6 mice were injected intragastrically with 12.5 mg/kg of recombinant N1₁₀₋₁₄FC peptibody or IgG Fc for three days postnatally (P1-P3). Shown is the quantification of post-natal day 5 (P5) retinal vasculature stained with Isolectin B4. Radial outgrowth and percent vascular coverage near the angiogenic front were reduced in N1₁₀₋₁₄Fc treated mice (N=7-8), while tip cell density and percent vascular coverage of the mature plexus were not statistically altered. Box-and-whisker plots show median, minimum, and maximum values. **P value <0.01, *P value <0.05.

### DETAILED DESCRIPTION OF THE INVENTION

This invention provides peptibody-based Notch inhibitors composed of the core binding domains, EGF-like repeats 10-14, of either Notch1 or Notch4. These Notch peptibodies showed high secretion properties and production yields when compared to previous Notch decoys. Using surface plasmon resonance spectroscopy coupled with co-immunoprecipitation assays, it was observed that Notch1 and Notch4 peptibodies demonstrate strong but distinct binding properties to Notch ligands DLL4 and JAG1. Both Notch1 and Notch4 peptibodies interfere with Notch signaling in endothelial cells and reduce expression of canonical Notch targets after treatment. While prior DLL4 inhibitors cause hyper-sprouting, the Notch1 peptibody reduced angiogenesis in a 3-dimensional in vitro sprouting assay. Administration of Notch1 peptibodies to neonate mice resulted in reduced radial outgrowth of retinal vasculature, confirming anti-angiogenic properties. Accordingly, this invention provides fusion proteins composed of human Notch1 and Notch4 extracellular domains, containing EGF-like repeats 10-14, fused to human IgG Fc (referred to herein as N1₁₀₋₁₄Fc and N4₁₀₋₁₄Fc) and methods of using the same to inhibit angiogenesis and treat an angiogenic disease or disorder.

Specifically, this invention provides a fusion protein, the sequence of which, commencing at the N-terminus of the fusion protein, is identical to the sequence of amino acids in: (a) an extracellular domain of a human Notch1 or Notch4 receptor protein, followed by (b) an Fc portion of an antibody, wherein the extracellular domain of the human Notch1 or Notch4 receptor protein (i) commences with the amino acid present at the N-terminus of EGF-like repeat 10 and (ii) extends to and includes the C-terminal amino acid of EGF-like repeat 14 as the C-terminal amino acid of the extracellular domain.

As used herein, a “fusion protein” or “chimeric protein” refers to a protein created through the joining of two or more proteins resulting in a single protein with the functional properties of the two or more proteins. The fusion protein of this invention comprises or consists of the EGF-like repeats 10-14 of Notch1 or Notch4 fused in frame to the Fc portion of an antibody. In certain aspects, the fusion protein is that set forth under SEQ ID NO:1 or SEQ ID NO: 3.

As used herein, “Notch,” “Notch protein,” and “Notch receptor protein” are synonymous. In addition, the terms “Notch-based fusion protein” and “Notch decoy” are synonymous. The following Notch amino acid sequences are known and readily available: Notch1 (GENBANK accession no. S18188 (rat)); Notch2 (GENBANK accession no. NP_077334 (rat)); Notch3 (GENBANK accession no. 061982 (mouse)); and Notch4 (GENBANK accession no. T09059 (mouse)). The following Notch nucleic acid sequences are known and readily available: Notch1 (GENBANK accession no. XM_342392 (rat) and NM_017617 (human)); Notch2 (GENBANK accession no. NM 024358 (rat), M99437 (human and (human)); AF308601 Notch3 (GENBANK accession no. NM 008716 (mouse) and XM_009303 (human)); and Notch4 (GENBANK accession no. NM_010929 (mouse) and NM_004557 (human)). In some aspects, the fusion protein of this invention includes a mammalian Notch1 or mammalian Notch4 extracellular domain containing EGF-like repeats 10-14, or more preferably a mammalian Notch1 extracellular domain containing EGF-like repeats 10-14. In other aspects, the fusion protein of this invention includes a human Notch1 or human Notch4 extracellular domain containing EGF-like repeats 10-14, or more preferably a human Notch1 extracellular domain containing EGF-like repeats 10-14. In further aspects, the fusion protein of this invention includes a human Notch1 extracellular domain containing EGF-like repeats 10-14 comprising or consisting of SEQ ID NO:5 or a human Notch4 extracellular domain containing EGF-like repeats 10-14 comprising or consisting of SEQ ID NO: 6.

In some aspects, the Notch1 extracellular domain containing EGF-like repeats 10-14 or Notch4 extracellular domain containing EGF-like repeats 10-14 does not include a signal sequence. Exemplary Notch1 and Notch4 extracellular domains containing EGF-like repeats 10-14 that do not include a signal sequence are respectively set forth in SEQ ID NO: 24 and SEQ ID NO:25. In other aspects, the Notch1 or Notch4 extracellular domain containing EGF-like repeats 10-14 includes a signal sequence. Exemplary Notch1 and Notch4 proteins with signal sequences are respectively set forth under SEQ ID NO: 5 and SEQ ID NO: 6.

As used herein, an Fc portion of an antibody encompasses domains derived from the constant region of an immunoglobulin, preferably a human immunoglobulin, including a fragment, analog, variant, mutant or derivative of the constant region. Suitable immunoglobulins include IgG1, IgG2, IgG3, IgG4, and other classes such as IgA, IgD, IgE and IgM. IgG subclasses are well known to those in the art and include, but are not limited to, human IgG1, IgG2, IgG3 and IgG4.

The “Fc portion of an antibody,” “Fc region,” and “Fc domain” interchangeably refer to a crystallizable fragment obtained by papain digestion of an immunoglobulin and includes all, or substantially all, of one C-terminal half of a heavy chain. For example, the Fc portion may include: a CH2 domain, a CH3 domain, a CH4 domain, a CH2-CH3 domain, a CH2-CH4 domain, a CH2-CH3-CH4 domain, a hinge-CH2 domain, a hinge-CH2-CH3 domain, a hinge-CH2-CH4 domain, or a hinge-CH2-CH3-CH4 domain. The Fc domain may be derived from antibodies belonging any of the immunoglobulin classes, i.e., IgA, IgD, IgE, IgG, or IgM or any of the IgG antibody subclasses, i.e., IgG1, IgG2, IgG3, and IgG4. The Fc domain may be a naturally occurring Fc sequence, including natural allelic or splice variants. Alternatively, the Fc domain may be a hybrid domain comprising a portion of an Fc domain from two or more different Ig isotypes, for example, an IgG2/IgG4 hybrid Fc domain. In a particular aspect, the Fc domain is derived from a human immunoglobulin molecule. Alternatively, the Fc domain may be a humanized or deimmunized version of an Fc domain from a non-human animal, including but not limited to mouse, rat, rabbit, camel, llama, dromedary and monkey.

Shown below is the sequence of a human IgG1 immunoglobulin constant region:

(SEQ ID NO: 7)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGPSVFLFPPKPKDTLMI
SRTPEVTCVVVDVSHEDPEVKENWYVDGVEVHNAKTKPREEQYNS
TYRVVSVLTVLHQDWLNGKEYKCKVSNKALPAPIEKTISKAKGQP
REPQVYTLPPSRDELTKNQVSLTCLVKGFYPSDIAVEWESNGQPE
NNYKTTPPVLDSDGSFFLYSKLTVDKSRWQQGNVFSCSVMHEALH
NHYTQKSLSLSPGK,

wherein the core hinge sequence is underlined. It should be understood that the C-terminal lysine is optional. Accordingly, the C-terminal lysine of an IgG sequence may be removed or replaced with a non-lysine amino acid, such as alanine, to further increase the serum half-life of the Fc fusion protein.

In one aspect, the hinge sequence may include substitutions that confer desirable pharmacokinetic, biophysical, and/or biological properties. Some exemplary hinge sequences are presented in Table 1.

TABLE 1

| Hinge Sequence* | SEQ ID NO: |
|---|---|
| EPKSSDKTHTCPPCPAPELLGGPS | 8 |
| EPKSSDKTHTCPPCPAPELLGGSS | 9 |
| EPKSSGSTHTCPPCPAPELLGGSS | 10 |
| DKTHTCPPCPAPELLGGPS | 11 |
| DKTHTCPPCPAPELLGGSS | 12 |

*core hinge region underlined.

In some aspects, the Fc domain is a variant Fc sequence, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, one may make modifications in the Fc region in order to generate an Fc variant that (a) has increased or decreased antibody-dependent cell-mediated cytotoxicity (ADCC), (b) increased or decreased complement mediated cytotoxicity (CDC), (c) has increased or decreased affinity for C1q and/or (d) has increased or decreased affinity for a Fc receptor relative to the parent Fc. Such Fc region variants will generally include at least one amino acid modification in the Fc region. Combining amino acid modifications is thought to be particularly desirable.

In other aspects, an Fc fusion protein described herein includes the CH2 and CH3 regions of a human IgG1 as shown below:

(SEQ ID NO: 13)
VFLFPPKPKDTLMISRTPEVTCVVVDVSHEDPEVKENWYVDGVEV
HNAKTKPREEQYNSTYRVVSVLTVLHQDWLNGKEYKCKVSNKALP
APIEKTISKAKGQPREPQVYTLPPSREEMTKNQVSLTCLVKGFYP
SDIAVEWESNGQPENNYKTTPPVLDSDGSFFLYSKLTVDKSRWQQ
GNVFSCSVMHEALHNHYTQKSLSLSPGK.

In some aspects, a fusion protein described herein includes an Fc domain that is at least 50%, 60%, 75%, 80%, 85%, 90%, 95%, 96%, 97%, 98%, or 99% identical to SEQ ID NO: 13. In other aspects, a fusion protein described herein includes an Fc domain of SEQ ID NO:13 with from 1-5, 1-10, 1-15, 1-20, or 1-25 substitutions or conservative substitutions. Fc variants may include those described below, wherein based upon the numbering scheme of the EU index as in Kabat et al. (1991) NIH Publication 91-3242, National Technical Information Service, Springfield, VA. Such variants include, but are not limited to, an Fc region having one or more of amino acid residues 234, 235, 236, 237, 297, 318, 320 and 322 substituted to a different amino acid residue such that the variant Fc region has an altered affinity for an effector ligand, e.g., an Fc receptor or the C1 component of complement, as described in U.S. Pat. Nos. 5,624,821 and 5,648,260, incorporated herein by reference; an Fc region having one or more of amino acid residues 329, 331 and 322 replaced such that the variant Fc region has altered C1q binding and/or reduced or abolished complement dependent cytotoxicity (CDC) as described in U.S. Pat. No. 6,194,551, incorporated herein by reference; an Fc region having one or more of amino acid residues 252, 254, 256, 433, 435, and 436 mutated to increase the serum half-life of the fusion protein as described in U.S. Pat. No. 6,277,375, incorporated herein by reference; and/or an Fc region having substitutions at positions 259, 308, 428, and 434 to increase binding to FcRn and/or improve pharmacokinetic properties.

In some aspects, hybrid IgG isotypes with particular biological characteristics may be used. For example, an IgG1/IgG3 or IgG1/IgG2 hybrid variant may be constructed by substituting IgG1 positions in the CH2 and/or CH3 region with the amino acids from IgG2 or IgG3 at positions where the two isotypes differ.

In other aspects, the glycosylation of the Fc is modified. Oligosaccharides that are covalently attached to the Fc region can be changed, for example by expressing an IgG in various organisms or cell lines, engineered or otherwise (for example Lec-13 CHO cells or rat hybridoma YB2/0 cells), by regulating enzymes involved in the glycosylation pathway (for example FUT8 [α1, 6-fucosyltranserase] and/or β1-4-N-acetylglucosaminyltransferase III [GnTIII]), by modifying carbohydrate(s) after the IgG has been expressed, or by expressing a fusion protein in the presence of fucose analogs as enzymatic inhibitors. Other methods for modifying glycoforms of Fc-containing fusion proteins include using glycoengineered strains of yeast (Li et al. (2006) Nature Biotechnology 24 (2): 210-215) or plants (Cox et al. (2006) Nat. Biotechnol. 24 (12): 1591-7).

In some aspects, the fusion protein does not include a linker between the Notch1 or Notch4 extracellular domain containing EGF-like repeats 10-14 and the Fc portion of the antibody. For example, a fusion protein may comprise an Fc domain that is linked directly to the Notch1 or Notch4 extracellular domain containing EGF-like repeats 10-14 without an intervening sequence.

In other aspects, a linker or adapter molecule composed of from 1-5 or 1-20 amino acids may be inserted between the Fc domain and the Notch1 or Notch4 extracellular domain containing EGF-like repeats 10-14. Exemplary linkers include, but are not limited to, DLGPG (SEQ ID NO:14), GSGSGSGSGSGS (SEQ ID NO: 15), AGGGGSG (SEQ ID NO:16), AGGGGSGG (SEQ ID NO: 17), QPDEPGGS (SEQ ID NO: 18), ELOLEESAAEAQDGELD (SEQ ID NO:19), TVAAPS (SEQ ID NO:20), QPDEPGGSG (SEQ ID NO: 21), or ELQLEESAAEAQDGELDG (SEQ ID NO: 22), TVAAPSG (SEQ ID NO: 23). In some, the linker comprises, consists essentially of, or consists of an amino acid sequence that is at least 50%, 60%, 75%, 80%, 85%, 90%, 95%, 96%, 97%, 98%, or 99% to any one of SEQ ID NOs: 14-23. In some aspects, the linker sequence does not contain a cysteine residue. A linker or adapter molecule may also be designed with a cleavage site for a protease to allow for the separation of the fused moieties.

The fusion protein of the invention may be chemically synthesized or be prepared by recombinant DNA techniques. Thus, in another aspect, the invention provides a nucleic acid molecule that encode the fusion protein of the invention. Exemplary nucleic acid molecules are set forth herein in SEQ ID NO: 2 and SEQ ID NO: 4. In a related aspect, the invention provides vectors, particularly expression vectors that comprise the nucleic acid molecule encoding the fusion protein. In some aspects, the vector provides replication, transcription and/or translation regulatory sequences that facilitate recombinant synthesis of the fusion protein in a eukaryotic cell or Accordingly, the invention also provides host cells for recombinant expression of the fusion protein and methods of harvesting and purifying the fusion protein produced by the host cells. Production and purification of recombinant proteins are routinely practiced by persons having ordinary skill in the art. The fusion protein can be purified by any suitable method known in the art including without limitation gel filtration and/or affinity purification. In some aspects, the fusion protein further includes a tag, e.g., an epitope tag, to facilitate purification, wherein the tag can optionally be cleaved off using a protease before further analysis.

Alternatively, the fusion protein of the invention can be synthesized by any of the chemical synthesis techniques known in the art, particularly solid-phase synthesis techniques, for example, using commercially-available automated peptide synthesizers. See, for example, Stewart & Young (1984) Solid Phase Peptide Synthesis, 2^{nd} Ed., Pierce Chemical Co.; Tam et al. (1983) J. Am. Chem. Soc. 105:6442; Merrifield (1986) Science 232:341-347; Barany et al. (1987) Int. J. Peptide Protein Res. 30:705-739; and U.S. Pat. No. 5,424,398.

Angiogenesis is believed to involve a complex interplay of molecules which both stimulate and inhibit the growth of endothelial cells, the primary cells of the capillary blood vessels. Under normal conditions, these molecules appear to maintain the microvasculature in a quiescent state (i.e., one of no capillary growth) for prolonged periods. When necessary, however (such as during wound repair), these cells can undergo rapid proliferation and turnover within a short period of time. Although angiogenesis is a highly regulated process under normal conditions, many conditions (characterized as “angiogenic diseases”) are driven by persistent unregulated angiogenesis. Otherwise stated, unregulated angiogenesis may either cause a particular pathological condition directly or exacerbate an existing pathological condition. There has been considerable evidence in vivo, including clinical observations, that abnormal angiogenesis is implicated in a number of disease conditions, which include rheumatoid arthritis, inflammation, cancer, psoriasis, degenerative eye conditions and others.

As demonstrated herein, the fusion protein of the invention exhibits anti-angiogenic activity. Accordingly, in one aspect, this invention further provides a method for inhibiting angiogenesis by contacting a cell or tissue with an effective amount of the fusion protein of the invention to inhibit angiogenesis. In accordance with this aspect, the cell may be an endothelial cell and the tissue may be tissue including endothelial cells and/or exhibiting excessive vascularity. Examples of such tissues include, but are not limited to ocular tissues, tumor tissues, and joint tissue. An effective amount of the fusion protein is that which: provides a measurable decrease in angiogenesis (e.g., 10%, 20%, 30%, 40%, 50%, 60%, 70%, 80%, 90% or 100% decrease) as compared to the same cell or tissue which has not been contacted with the fusion protein. The inhibition or decrease in angiogenesis can be assessed as demonstrated herein by measuring cleavage of endogenous Notch1 expressed on the surface of endothelial cells, determining the expression of Notch target genes NRARP, HEY2, RND1, and/or DLL4, and/or assessing endothelial sprout formation and/or length.

The fusion protein of t this invention also finds application in the treatment of angiogenic diseases or disorders by inhibiting Notch signaling. Accordingly, another aspect of this invention provides a method for treating an angiogenic disease or disorder in a subject by administering to the subject an effective amount of the fusion protein of the invention. As used herein, “treating,” “treatment,” or “to treat” each may mean to alleviate, suppress, repress, eliminate, or slow the appearance of symptoms, clinical signs, or underlying pathology of a condition or disorder on a temporary or permanent basis. Suppressing a condition or disorder involves administering a fusion protein of the present invention to a subject after induction of the disease or disorder but before its clinical appearance. Repressing the disease or disorder involves administering a fusion protein of the present invention to a subject after clinical appearance of the disease or disorder.

For the purposes of this invention, an “angiogenic disease or disorder” is characterized by persistent unregulated or abnormal angiogenesis resulting in excessive vascularity. Angiogeneic diseases are also characterized by having a high level of vascular inflammation or in response to a high level of inflammation in tissues. For example, ocular neovascularization has been implicated as the most common cause of blindness. In certain existing conditions such as arthritis, newly formed capillary blood vessels invade the joints and destroy cartilage. In diabetes, new capillaries formed in the retina invade the vitreous, bleed, and cause blindness. Growth and metastasis of solid tumors are also angiogenesis-dependent (Folkman (1986) Cancer Res. 46:467-473; Folkman (1989) J. Natl. Cancer Inst. 82:4-6). It has been shown, for example, that tumors which enlarge to greater than 2 mm must obtain their own blood supply and do so by inducing the growth of new capillary blood vessels. Once these new blood vessels become embedded in the tumor, they provide a means for tumor cells to enter the circulation and metastasize to distant sites, such as the liver, the lung, and the bones (Weidner et. al. (1991) N. Engl. J. Med. 324 (1): 1-8).

Thus, the fusion protein of this invention may be used in the treatment of angiogenic diseases or disorders including, but not limited to, atherosclerosis, wound healing, ocular diseases or disorders, pre-eclampsia, ischemia, stroke, cardiovascular disease, psoriasis, lymphedema, vascular inflammation or cancer.

In some aspects, this invention provides a method for the treatment of an ocular disease or condition. In particular, the fusion protein is of use in the treatment of ocular diseases or disorders such as retinopathy of prematurity, corneal graft rejection, retrolental fibroplasia, neovascular glaucoma, rubeosis, proliferative diabetic retinopathy, ischemic retinopathy, intraocular neovascularization, corneal neovascularization, retinal neovascularization, choroidal neovascularization, diabetic macular edema, diabetic retina ischemia, diabetic retinal edema, neovascular age-related macular degeneration (AMD), central retinal vein occlusion, branch retinal venin occlusion, retinitis pigmentosa, rubeosis iridis, visual impairment or vision loss (blindness) associated with angiogenesis, retinoblastoma, uveitis and corneal graft neovascularization, angiogenesis in the eye associated with infection or surgical intervention, and other abnormal neovascularization conditions of the eye. In other aspects, the ocular disease or disorder is AMD or diabetic retinopathy.

In particular aspects, the AMD is wet or dry AMD. Wet macular degeneration occurs when abnormal blood vessels grow behind the macula. These vessels are fragile and can leak fluid and blood, which result in scarring of the macula and raise the potential for rapid, severe damage. Bruch's membrane breaks down, usually near drusen deposits. This is where new blood vessel growth, or neovascularization, occurs. Central vision can become distorted or lost entirely in a short period of time, sometimes within days. Thus, treatment in accordance with the method of this invention would ameliorate or decrease blood vessel growth or neovascularization and reduce loss of vision.

In some aspects, this invention provides a method for the treatment of cancers. Cancers that may be treated include, but are not limited to, primary and metastatic solid tumors, including carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). The fusion protein is also useful in treating solid tumors arising from hematopoietic malignancies such as leukemias (i.e., chloromas, plasmacytomas and the plaques and tumors of mycosis fungosides and cutaneous T-cell lymphoma/leukemia) as well as in the treatment of lymphomas (both Hodgkin's and non-Hodgkin's lymphomas). In addition, the fusion protein may be used alone or in combination with radiotherapy and/or other chemotherapeutic agents.

Further uses of the fusion protein include the treatment of autoimmune diseases such as rheumatoid, immune and degenerative arthritis; skin diseases such as psoriasis; blood vessel diseases such as hemagiomas, and capillary proliferation within atherosclerotic plaques; pulmonary fibrosis; Osler-Webber Syndrome; myocardial angiogenesis; asthma; plaque neovascularization; telangiectasia; hemophiliac joints; angiofibroma; ocular diseases and wound granulation. Other uses include the treatment of diseases characterized by excessive or abnormal stimulation of endothelial cells, including not limited to intestinal adhesions, Crohn's disease, atherosclerosis, scleroderma, and hypertrophic scars, i.e., keloids. Another use is in treatment of neuroinflammatory diseases, including infection-induced neuroinflammation and Alzheimer Disease (AD). Another use is as a birth control agent, by inhibiting ovulation and establishment of the placenta. The fusion protein of the invention may also be useful in the treatment of diseases that have angiogenesis as a pathologic consequence such as cat scratch disease and ulcers (Helicobacter pylori).

The fusion protein may further be useful in the treatment of Metabolic Syndrome, which is a combination of medical disorders that increase the risk for cardiovascular disease and diabetes. Other known names referring to such syndrome is syndrome X, insulin resistance syndrome, Reaven's syndrome. Several features of the syndromes include fasting hyperglycemia, high blood pressure, central obesity (also known as visceral obesity), decreased High Density Lipoprotein (LDL), elevated triglycerides, elevated uric acid levels. Fasting hyperglycemia, listed above, includes diabetes mellitus type 2 or impaired fasting glucose and impaired glucose tolerance or insulin resistance. In addition to metabolic syndrome, the Notch decoy may have indications for pre-diabetic states.

Methods of treatment in accordance with this invention are carried out by administering to a subject, having a disease or disorder characterized by excessive or abnormal vascularity, an effective amount of the fusion protein, or pharmaceutical composition containing the same. As used herein, the term “administration” or “administering” refers to the process of delivering an agent to a patient. The process of administration can be varied, depending on the agent, or agents, and the desired effect.

The term “effective amount” or “therapeutically effective amount” depends on the condition of a subject and the specific fusion protein administered. The term refers to an amount effective to achieve, a desired clinical effect. An effective amount varies with the nature of the condition being treated, the length of time that activity is desired, and the age and the condition of the subject, and ultimately is determined by the health care provider. In general, however, doses employed for adult human treatment typically are in the range of 0.001 mg/kg to about 200 mg/kg per day. The dose may be about 0.05 mg/kg to about 10 g/kg per day. The desired dose may be conveniently administered in a single dose, or as multiple doses administered at appropriate intervals, for example as two, three, four or more sub-doses per day. Multiple doses may be desired, or required.

The subject being treated may be a mammal, which may be a human. Prior to diagnosis, the subject may be at risk for an angiogenic disease or disorder because of exposure to one or more risk factors or have a genetic risk for developing an angiogenic disease or disorder. By way of illustration, the one or more risk factors for cancer may include, for example, the subject having a family history of cancer, age, smoking tobacco, sun exposure, drinking alcoholic beverages, lack of physical activity, obesity and/or dietary deficiency. Illustrative examples of risk factors for developing macular degeneration include, for example, the subject having a family history of macular degeneration, age, smoking tobacco, prolonged sun exposure, high fat diet, dietary deficiency, high blood pressure, obesity, and/or light color eyes.

Suitable methods of administering a fusion protein, such as a pharmaceutical composition including the fusion protein described herein, are well known in the art. Although more than one route can be used to administer a fusion protein, a particular route can provide a more immediate and more effective reaction than another route. Depending on the circumstances, a pharmaceutical composition comprising the fusion protein is applied or instilled into body cavities, absorbed through the skin or mucous membranes, ingested, inhaled, and/or introduced into circulation. For example, in certain circumstances, it will be desirable to deliver the pharmaceutical composition orally; through injection or infusion by intravenous, intratumoral, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means; by controlled, delayed, sustained or otherwise modified release systems; or by implantation devices. In one aspect, drug exposure can be optimized by maintaining constant drug plasma concentrations over time. Such a steady-state is generally accomplished in clinical settings by continuous drug infusion at doses depending on the drug clearance and the plasma concentration to be sustained. If desired, the composition is administered regionally via intratumoral, administration, intrathecal administration, intracerebral (intra-parenchymal) administration, intracerebroventricular administration, or intraarterial or intravenous administration targeting the region of interest. Alternatively, the fusion protein is administered locally via implantation of a matrix, membrane, sponge, or another appropriate material onto which the desired compound has been absorbed or encapsulated. Where an implantation device is used, the device is, in one aspect, implanted into any suitable tissue or organ, and delivery of the fusion protein is, for example, via diffusion, timed-release bolus, or continuous administration.

Ocular administration of the fusion protein may be carried using intraocular implants, intravitreal injections, systemic administration, topical application, nanoparticles, microparticles, eye drops, bioadhesive gels or fibrin sealant, polysaccharides to modulate the permeability of the epithelial cell barrier complex, peptide enhances corneal drug delivery, mucosal administration such as administration using a biovector polymer, aqueous opthamalic sprays and electrodynamic ocular spray treatment. In one particular aspect, the fusion protein may be administered by intravitreal injection or topically such as in the form of an eye drop.

The fusion protein may be administered as monotherapy or simultaneously or metronomically with other treatments, which may be a surgery or removal of a tumor. The term “simultaneous” or “simultaneously” as used herein, means that the fusion protein and other treatment be administered within 48 hours, preferably 24 hours, more preferably 12 hours, yet more preferably 6 hours, and most preferably 3 hours or less, of each other. The term “metronomically” as used herein means the administration of the fusion protein at times different from the other treatment and at a certain frequency relative to repeat administration. For example, the fusion protein of the invention may be administered with one or more VEGF inhibitors. For example, the fusion protein of the invention may be administered with one or more VEGF inhibitors or in combination with laser treatment for vision loss.

Fusion proteins provided herein may be in the form pharmaceutical compositions including the fusion protein and a pharmaceutically acceptable excipient. The fusion protein may be formulated in the form of tablets or lozenges formulated in a conventional manner. For example, tablets and capsules for oral administration may contain conventional excipients may be binding agents, fillers, lubricants, disintegrants and wetting agents. Binding agents include, but are not limited to, syrup, acacia, gelatin, sorbitol, tragacanth, mucilage of starch and polyvinylpyrrolidone. Fillers may be lactose, sugar, microcrystalline cellulose, maize starch, calcium phosphate, and sorbitol. Lubricants include, but are not limited to, magnesium stearate, stearic acid, talc, polyethylene glycol, and silica. Disintegrants may be potato starch and sodium starch glycollate. Wetting agents may be sodium lauryl sulfate. Tablets may be coated according to methods well known in the art.

The fusion protein provided herein may also be liquid formulations such as aqueous or oily suspensions, solutions, emulsions, syrups, and elixirs. The fusion protein may also be formulated as a dry product for constitution with water or other suitable vehicle before use. Such liquid preparations may contain additives such as suspending agents, emulsifying agents, nonaqueous vehicles and preservatives. Suspending agent may be sorbitol syrup, methyl cellulose, glucose/sugar syrup, gelatin, hydroxyethyl cellulose, carboxymethyl cellulose, aluminum stearate gel, and hydrogenated edible fats. Emulsifying agents may be lecithin, sorbitan monooleate, and acacia. Nonaqueous vehicles may be edible oils, almond oil, fractionated coconut oil, oily esters, propylene glycol, and ethyl alcohol. Preservatives may be methyl or propyl p-hydroxybenzoate and sorbic acid. In particular, the fusion protein of the invention may be in aqueous formulations for topical administration such as in the form of an eye drop.

The fusion protein provided herein may also be formulated as a suppository, which may contain suppository bases such as cocoa butter or glycerides. The fusion protein provided herein may also be formulated for inhalation, which may be in a form such as a solution, suspension, or emulsion that may be administered as a dry powder or in the form of an aerosol using a propellant, such as dichlorodifluoromethane or trichlorofluoromethane. The fusion protein provided herein may also be formulated as transdermal formulations comprising aqueous or nonaqueous vehicles such as creams, ointments, lotions, pastes, medicated plaster, patch, or membrane. The fusion protein provided herein may also be formulated for parenteral administration such as by injection, intratumor injection or continuous infusion. Formulations for injection may be in the form of suspensions, solutions, or emulsions in oily or aqueous vehicles, and may contain formulation agents including, but not limited to, suspending, stabilizing, and dispersing agents. The fusion protein may also be provided in a powder form for reconstitution with a suitable vehicle including, but not limited to, sterile, pyrogen-free water.

The fusion protein provided herein may also be formulated as a depot preparation, which may be administered by implantation or by intramuscular injection. The fusion protein may be formulated with suitable polymeric or hydrophobic materials (as an emulsion in an acceptable oil, for example), ion exchange resins, or as sparingly soluble derivatives (as a sparingly soluble salt, for example).

The foregoing may be better understood by reference to the following discussion and results which are presented for purposes of illustration and are not intended to limit the scope of the invention.

### Example 1: Materials and Methods

Expression and Purification of Notch Decoys. Expression vectors of N1₁₀₋₁₄Fc and N4₁₀₋₁₄Fc were transfected in HEK EXPI293™ cells using the EXPI293™ Expression System (ThermoFisher Scientific). Notch decoys were subsequently purified from cultured media by HITRAP® rProtein A FF (GE Healthcare) affinity chromatography. Eluted fractions were collected and immediately dialyzed to exchange buffer into phosphate-buffered saline. Protein was concentrated in VIVASPIN® 20 10,000 MWCO centrifugal concentrators (Sartorius).

Cell Lines. All cell cultures were maintained at 37° C. in a mixture of 5% CO₂ and 95% humidified air. HUVECs isolated from human umbilical veins (Lonza) were grown in EGM-2 Medium (Lonza) on culture plates coated with rat tail type I collagen (BD Biosciences). HEK293T cells were purchased from ATCC and maintained on Dulbecco's Modified Eagle Medium (DMEM, Gibco) with 10% bovine fetal serum (FBS). Normal human lung fibroblasts (NHLFB) were purchased from Lonza and cultured with fibroblast growth media (Lonza).

Western Blot Analysis. Cells were lysed in ice cold cell RIPA buffer (Cell Signaling) containing 1× protease inhibitor (ThermoFisher Scientific), 1× phosphatase inhibitor (ThermoFisher Scientific), and 1 mM of DDT, and western blot analysis was performed. Primary antibodies against cleaved Notch1, protein tag sold under the tradename FLAG®, MYC, and Actin were from Cell Signaling Technology and incubated in blocking buffer (5% BSA, and 1×TBST (0.1% polysorbate 20)). Gel images were obtained using the CHEMIDOCT MP imaging system (Bio-Rad), and quantitation was performed using ImageJ.

Co-Immunoprecipitation Assay. N1₁₀₋₁₄Fc or N4₁₀₋₁₄Fc and full-length DLL4-MYC or JAG1-FLAG® were transiently co-transfected into HEK-293T cells using the transfection reagent sold under the tradename LIPOFECTAMINE® 2000. A crosslinking agent, disuccinimidyl glutarate (ThermoFisher Scientific), was added to the culture 24 hours after transfection at a final concentration of 20 nmol/ml and incubated for 30 minutes. The cells were subsequently lysed in 100 μl of 1× cell lysis buffer from Cell Signaling. The lysate was pulled down by 20 μl of Protein A/G magnetic beads (ThermoFisher Scientific). To reverse the crosslink prior to western blot analysis, the immunocomplex was treated with 50 μmol/ml dithiothreitol (DTT) and boiled for 4 minutes before electrophoresis.

Affinity Analysis. The binding kinetics of N1₁₀₋₁₄Fc and N4₁₀₋₁₄Fc were analyzed using a Surface Plasmon Resonance (SPR)-based assay on the BIACORE™ T200 system (GE Healthcare). Human IgG Fc (Sino Biologics) was first immobilized on a CM5 biosensor chip. Subsequently, an appropriate concentration of hDLL4-Fc (Sino Biologics) and hJAG1-Fc (Sino Biologics) was captured to the surface at a Response Unit (RU) of up to 20,000. Various concentrations of N1₁₀₋₁₄Fc and N4₁₀₋₁₄Fc were then passed through the chip with running buffer (1×HBS-N (10 mM HEPES, 150 mM NaCl) with 0.005% polysorbate 20, 1 mM CaCl₂), 2 mM MgCl₂, PH 7.4). After each reaction, the captured ligands and analyte were removed by regeneration buffer (1×HBS-N (10 mM HEPES, 150 mM NaCl) with 0.005% polysorbate 20, 1 mM CaCl₂), 2 mM MgCl₂, pH 7.4). The whole reaction was conducted at 25° C. and a flow rate of 25 μl/min. Sensorgrams of each concentration were obtained and analyzed by BIACORE™ evaluation software (GE Healthcare). The equilibrium constant K_{D} was calculated from the ratio of the dissociation rate constant k_{d} to the association rate constant kₐ (k_{d}/kₐ).

Quantitative Real-Time Polymerase Chain Reaction (qRT-PCR). Total RNA from HUVECs treated with either human IgG Fc (Sino Biologics), N1₁₀₋₁₄Fc or N4₁₀₋₁₄Fc was collected after 24 hours as recommended by the manufacturer using Qiagen RNEASY®. Complementary DNA (cDNA) synthesis was performed using approximately 1 μg RNA per 20 μl using a cDNA reverse transcription kit (ThermoFisher Scientific). Real-time PCR was performed on a VIIA™ 7 real time PCR system (Life Technologies) using a cyanine dye sold under the tradename SYBR™ Green.

Cell Viability Assay. 96-well plates were seeded with 4×10³ HUVEC cells (Lonza) and increasing concentrations of either N1₁₀₋₁₄Fc or N4₁₀₋₁₄Fc were added, each concentration having six repeats. Human IgG Fc (Sino Biologics) was added as control. After incubation for 72 hours, cell viability was determined by XTT assay (Biotium).

Scratch-Wound Healing Assay. HUVECs (Lonza) treated with CellTracker CMFDA dye (ThermoFisher) were seeded in 24-well plates coated with rat tail type I collagen (BD Biosciences) and “scratch-wounded” using a 200 μl pipette tip. After wounding, cells were treated with different concentrations (5 μ/μl or 10 μ/μl) of either IgG Fc, N1₁₀₋₁₄Fc or N4₁₀₋₁₄Fc. After approximately 14 hours, microscopy was used to image cell migration to the scratch.

Fibrin Bead Assay (FiBA). To evaluate the angiogenic potential of Notch peptibodies, 6×10⁴ HUVEC cells (Lonza) were used to coat 150 beads sold under the tradename CYTODEX® (Sigma) in endothelial growth media (EGM, Lonza). The endothelial-coated beads were embedded in fibrin gel (3 mg/ml) with either human IgG Fc (Sino Biologics), N1₁₀₋₁₄Fc, or N4₁₀₋₁₄Fc. 5×10⁴ NHLFB were seeded on top of the fibrin gel in EGM. The medium was changed every other day until day 12. The sprout numbers and length were analyzed by Image J (NIH).

Mice. All mice used in this study were maintained in a pure C57BL/6J background. Male and female pups were used arbitrarily in these studies.

Retinal Analysis. C57BL/6 mice postnatal day 1 (P1) pups were injected intragastrically with 12.5 mg/kg of recombinant N1₁₀₋₁₄Fc decoy or Fc for three days (P1-P3). Eyes were isolated at P5 and were fixed in 4% paraformaldehyde (ThermoFisher Scientific) for 1 hour at 4° C. on a nutator. Following fixation, eyes were washed with 1×PBS solution. Retinas were dissected and permeabilized in 1×PBS containing 1% bovine serum albumin (BSA, Fisher Bioreagents) and 0.5% TRITON™ X-100 (Fisher Bioreagents) overnight at 4° C. on a nutator. Samples were then immunostained in PBLEC (5% TRITON™ X-100, 1M MgCl₂, 1M CaCl₂), and 1M MnCl₂ in 1×PBS) overnight at 4° C. with biotinylated IB4 (1:50; Vector Laboratories) and anti-α-SMA-FITC (1:200; MilliporeSigma). IB4 was detected with streptavidin-conjugated with the fluorescent dye sold under the tradename FLUOR® ALEXA 647 (Invitrogen). Immunostained retinas were postfixed with 4% formaldehyde and mounted in mounting medium sold under the tradename VECTASHIELD® (Vector Laboratories). Whole-mount retina images were acquired using Leica Dmi8 Platform. All images were analyzed using ImageJ (NIH).

Statistics. For qRT-PCR analysis, the ΔΔCt method was used to calculate the relative expression using following steps: (1) Normalization to reference gene: ΔCt_{GOI}=Ct_{GOI}−Ct_{BA} and (2) Relative expression between conditions: ΔΔCt_{GOI}=ΔCt_{EXP}−ΔCt_{CNT}. Unless noted otherwise, t-tests analysis was performed on all quantified data to determine significant differences between groups using GraphPad Prism 9. P values less than 0.05 were considered statistically significant. p-values <0.05 are shown with one star (*), p values <0.01 with two stars (**), and p-values <0.001 with three stars (***). All experiments shown were repeated a minimum of three times.

### Example 2: Design and Characterization of Notch Peptibodies

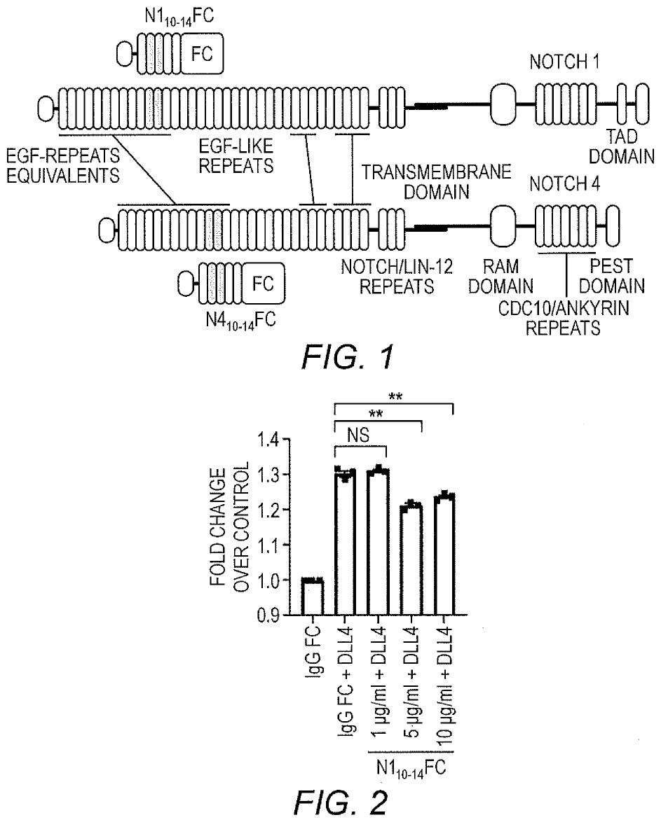

The extracellular domain of the four Notch proteins is composed of a varying number of EGF-like repeats. For Notch1, structure-function coupled with analysis high resolution crystal structures have identified EGF-like repeats 11-12 as critical for receptor-ligand interaction. In contrast to the Notch1 receptor, the core ligand-binding domain of Notch4, an endothelial-specific Notch gene, has not been fully characterized. While Notch4 is the most divergent of the four mammalian Notch receptors, EGFs 11-12 are highly homologous between Notch1 and Notch4. Of the four human Notch receptors, Notch1, 2, and 4 demonstrate high levels of conservation between EGF-like repeats 10-14 while a notable divergence in conservation appears in Notch3, where EGFs 7-10 of the Notch3 receptor best corresponds to EGFs 11-13 of Notch1. Further, protein alignment of the human and murine Notch receptors across EGFs 10-14 demonstrates high identity and similarity amongst species. Here, recombinant fragments of the human Notch1 Notch4 and extracellular domains containing the sequences of Notch1 and Notch4 EGF-like repeats 10-14 were fused to human IgG Fc. The resulting fusion proteins are referred to herein as “N1₁₀₋₁₄Fc” and “N4₁₀₋₁₄Fc” (FIG. 1).

N1₁₀₋₁₄Fc and N4₁₀₋₁₄Fc proteins were produced in HEK293F cells upon transfection with the corresponding expression construct. The secreted proteins were subsequently column purified and resolved in 4-20% SDS-PAGE gels stained with Coomassie, showing approximate molecular weights (MW) of N1₁₀₋₁₄Fc and N4₁₀₋₁₄Fc with an expected band at ˜50 kDa in reducing conditions. To confirm the identity of the proteins, western blot analysis was performed using antibodies specific for human IgG Fc in both non-reducing and reducing conditions, showing specific bands at ˜100 kDa in non-reducing and ˜50 kDa in reducing conditions. No evidence of cleavage between the Notch EGF-like repeats and Fc domain or other forms of degradation were apparent. As expected, detection of native protein under non-reducing conditions was at double the predicted molecular weight, indicating dimerization of the IgG Fc. To accurately assess their oligomeric states, mass photometry was used to evaluate N1₁₀₋₁₄Fc and N4₁₀₋₁₄Fc proteins. Both Notch decoys displayed an oligomeric mixture dominated by dimers at 100 kDa.

### Example 3: Both N1₁₀₋₁₄Fc and N4₁₀₋₁₄Fc Bind to DLL4 and JAG1

It was subsequently determined whether the homologous ligand-binding domain of Notch4 is sufficient to bind to Notch ligands DLL4 and JAG1. It has been shown that for Notch1, EGF-like repeats 11 and 12 correspond to the core binding domain and are alone sufficient for ligand interaction. There has been little evidence that Notch4 binds to ligands DLL4 and JAG1. To confirm binding specificity, N1₁₀₋₁₄Fc, N4₁₀₋₁₄Fc, full-length FLAG®-tagged JAG1, and full-length Myc-tagged DLL4 were co-expressed in 293T and cells co-immunoprecipitation was performed with Notch decoys acting as the “bait” protein. After pulldown, N1₁₀₋₁₄Fc and N4₁₀₋₁₄Fc co-immunoprecipitated with both DLL4 and JAG1, validating pan-ligand binding.

Surface plasmon resonance (SPR) spectroscopy was used to characterize and quantify the interactions between these proteins. Recombinant Fc-tagged hDLL4 and hJAG1 were immobilized on the sensor chip using amine coupling and multi-cycle kinetic experiments were performed using increasing concentrations of either N1₁₀₋₁₄Fc or N4₁₀₋₁₄Fc. As a control, recombinant IgG Fc was immobilized on the sensor chip. Recombinant hDLL4 interacted with N1₁₀₋₁₄Fc and N4₁₀₋₁₄Fc with the dissociation constants (K_{D}) provided in Table 2.

TABLE 2
Binding

| | | |
|---|---|---|
| Characteristic | N1₁₀₋₁₄Fc | N4₁₀₋₁₄Fc |

| | | | | |
|---|---|---|---|---|
| kₐ | 3.15 × 10⁴ | (M⁻¹s⁻¹) | 1.03 × 10⁴ | (M⁻¹s⁻¹) |
| k_{d} | 1.9 × 10⁻² | (s−1) | 9.9 × 10⁻³ | (s−1) |

| | | |
|---|---|---|
| K_{D} | 6.05 × 10⁻⁷M | 9.625 × 10⁻⁷M |
| Rₘₐₓ | 352.1 | 318.9 |
| Chi² | 8.76 | 1.94 |

As expected, N1₁₀₋₁₄Fc bound to DLL4 and JAG1. As demonstrated herein, there is a conserved binding domain within Notch4 that promotes interaction with DLL4 and JAG1. A notable feature of the sensorgrams between the two decoys show N1₁₀₋₁₄Fc binding to hDLL4 at a fast association and disassociation rate compared to N4₁₀₋₁₄Fc, indicating different binding mechanics. Although no interaction was measured between hJAG1 and N1₁₀₋₁₄Fc or N4₁₀₋₁₄Fc in SPR-based binding assays, it has been suggested that the interaction of the Notch1 extracellular domain and that of JAG1 is weak and requires a pulling force to stabilize JAG1 into confirmations needed for interaction. It was concluded that the Notch decoys described herein can readily bind to members of Delta-like or Jagged/Serrate-class Notch ligands.

### Example 4: N1₁₀₋₁₄Fc Suppresses Endothelial Notch Signaling

Interaction of endothelial Notch with Notch ligands, such as DLL4, causes the cleavage of the intracellular domain of Notch1 and subsequent translocation to the nucleus. Notch decoys that inhibit DLL4-Notch interactions would be expected to inhibit cleavage of endogenous Notch1 expressed on the surface of endothelial cells. To evaluate whether Notch decoys can block DLL4-mediated Notch1 activation, HUVECs were seeded onto DLL4-coated plates and subsequently dosed with increasing concentrations of N1₁₀₋₁₄Fc. At the highest doses tested, N1₁₀₋₁₄Fc reduced the DLL4-induced cleavage of endogenous Notch1 expressed in HUVECs (FIG. 2).

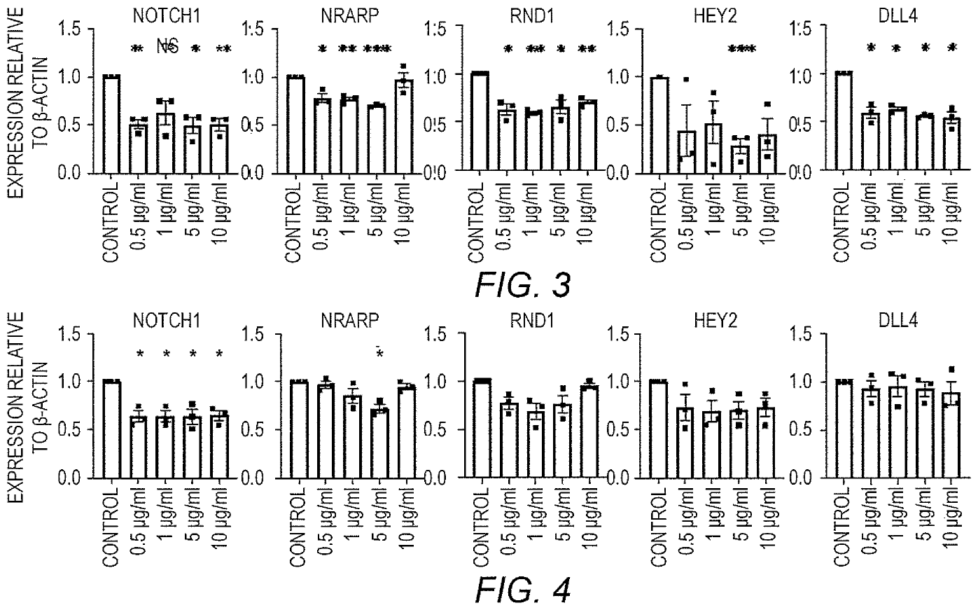

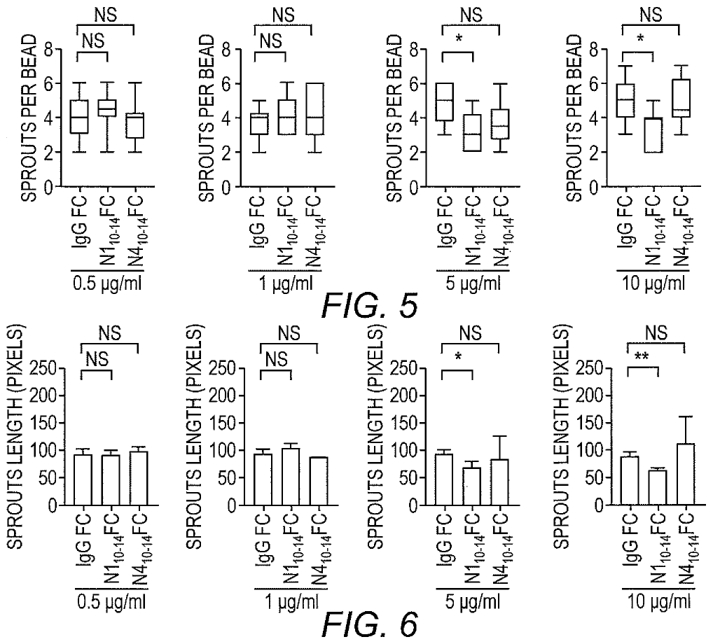

Once the NICD enters the nucleus, it interacts with the transcription factor RBPJ/CSL to regulate expression of canonical Notch target proteins. To directly test whether Notch decoys affect the canonical Notch signaling pathway, a crucial regulator in endothelial cells, the inhibitory effects of N1₁₀₋₁₄Fc and N4₁₀₋₁₄Fc on the Notch pathway in HUVECs were examined. Increasing concentrations (0, 0.5, 1, 5 and 10 μg/ml) of either IgG Fc, N1₁₀₋₁₄Fc, or N4₁₀₋₁₄Fc were used to treat HUVECs for 24 hours. Differences in mRNA expression of Notch target genes were then examined by qRT-PCR. Compared to the control group, N1₁₀₋₁₄Fc significantly downregulated Notch target genes NRARP, HEY2, RND1, DLL4, and Notch1 at multiple concentrations (FIG. 3). N4₁₀₋₁₄Fc decoy downregulated Notch target genes to a lesser degree in HUVECs (FIG. 4).

### Example 5: N1₁₀₋₁₄Fc Inhibits Angiogenesis In Vitro

Angiogenesis is a tightly controlled, multi-step process in endothelial cells that involves proliferation, cell migration, and tube formation. To assess the effects of the Notch decoys on endothelial sprout formation, a three-dimensional in vitro assay was used. Beads sold under the tradename CYTODEX® were coated with HUVECs and subsequently embedded into a fibrinogen matrix. To support HUVEC growth, fibroblast cells were cultured on top of the matrix to provide growth factors. In this assay, endothelial sprouts grow out from the bead, mimicking the nascent stages of angiogenesis. The number of outgrowths and their corresponding length in HUVECs treated with increasing concentrations of either IgG Fc, N1₁₀₋₁₄Fc, or N4₁₀₋₁₄Fc were evaluated. After treatment with N1₁₀₋₁₄Fc, the number of angiogenic sprouts (FIG. 5) and the length of the newly formed sprouts (FIG. 6) were reduced at concentrations of 5 and 10 μg/ml of the Notch decoy N1₁₀₋₁₄Fc.

To assess whether Notch decoys have cytotoxic effects on HUVECs, cells were treated with increasing concentrations of either IgG Fc, N1₁₀₋₁₄Fc, or N4₁₀₋₁₄Fc for 72 hours. The viability of HUVECs was dose-dependently inhibited by N110-14Fc, but not by N4₁₀₋₁₄Fc.

It was subsequently determined whether the anti-angiogenic effect observed in vitro was due in part to a migration defect or solely viability. It has previously been demonstrated that inhibition of DLL4-Notch signaling induces the migration of endothelial cells. To understand the role of Notch decoys on endothelial cell migration, a scratch wound-healing assay was used in which the extent of migration of cells into the scratched areas was examined. HUVECs treated with either IgG Fc, N1₁₀₋₁₄Fc, or N4₁₀₋₁₄Fc were assessed and no migration defects were detected. These results indicate that N1₁₀₋₁₄Fc, but not N4₁₀₋₁₄Fc, is biologically modulating endothelial cell behavior.

### Example 6: N1₁₀₋₁₄Fc Inhibits Murine Retinal Angiogenesis

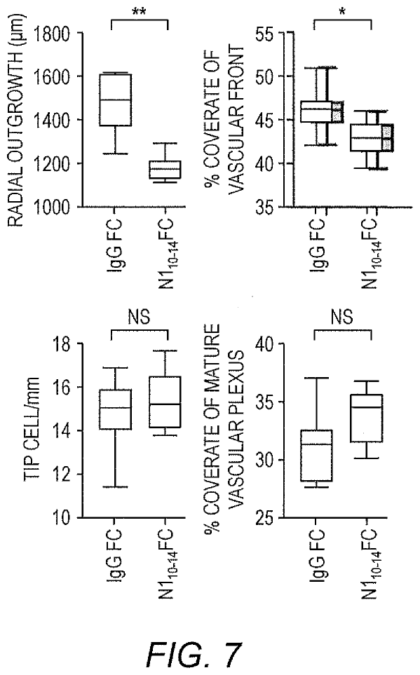

To better understand how peptibody-based Notch inhibitors effect angiogenesis in vivo, N1₁₀₋₁₄Fc treatment during postnatal murine angiogenesis was examined. To deliver the recombinant proteins, either human IgG Fc or N1₁₀₋₁₄Fc were injected intragastrically into murine neonates. Compared to the Fc-treated group, N1₁₀₋₁₄Fc showed a reduction in both the vascular area of the angiogenic front and radial vascular outgrowth (FIG. 7). Further, filopodia-extending endothelial sprouts, termed Tip Cells, were observed to be no more abundant in mice treated with N1₁₀₋₁₄Fc than Fc. While not the focus of this investigation, it was observed that approximately half the mice treated with N1₁₀₋₁₄Fc exhibited unusual enlargement of retinal veins.

In some vascular development settings, Notch ligands JAG1 and DLL4 play a crucial role in the recruitment of vascular smooth muscle cells to nascent arteries during the maturation process of angiogenesis. Vascular smooth muscle cell coverage of mice treated with N1₁₀₋₁₄Fc remained unchanged compared to the control group, indicating that N1₁₀₋₁₄Fc inhibited angiogenesis with no effect on vascular remodeling at this time point. These results indicate that N1₁₀₋₁₄Fc can cause inhibition of angiogenic vessels in vivo.

What is claimed is:

1. A fusion protein, the sequence of which, commencing at the N-terminus of the fusion protein, is identical to the sequence of amino acids in:
(a) an extracellular domain of a human Notch1 or Notch4 receptor protein, followed by
(b) an Fc portion of an antibody,
wherein the extracellular domain of the human Notch1 or Notch4 receptor protein
(i) commences with the amino acid present at the N-terminus of EGF-like repeat 10 and
(ii) extends to and includes the C-terminal amino acid of EGF-like repeat 14 as the C-terminal amino acid of the extracellular domain.

2. The fusion protein of claim 1, further comprising a signal sequence.

3. A nucleic acid molecule encoding the fusion protein of claim 1.

4. A host cell comprising the nucleic acid molecule of claim 3.

5. A pharmaceutical composition comprising the fusion protein of claim 1 and a pharmaceutically acceptable excipient.

6. A method of inhibiting angiogenesis comprising contacting a cell or tissue with an effective amount of the fusion protein of claim 1 thereby inhibiting angiogenesis.

7. A method of treating an angiogenic disease or disorder in a subject comprising administering to the subject an effective amount of the fusion protein of claim 1 thereby treating the subject's angiogenic disease or disorder.

8. The method of claim 7, wherein the angiogenic disease or disorder comprises an ocular disease or disorder.

9. The method of claim 8, wherein the ocular disease or disorder is age-related macular degeneration (AMD) or diabetic retinopathy.

10. The method of claim 7, wherein the angiogenic disease or disorder is cancer.

11. The method of claim 7, wherein the angiogenic disease or disorder is vascular inflammation.
